(19) United States
(12) Patent Application Publication
(10) Pub. No.: US 2023/0074841 A1
(43) Pub. Date: Mar. 9, 2023

(54) PROSTHETIC FOOT ASSEMBLY

(71) Applicant: Walter Sisulu University, Eastern Cape (ZA)

(72) Inventor: Zanodumo Thandazani Godlimpi, Eastern Cape (ZA)

(21) Appl. No.: 17/756,880

(22) PCT Filed: Dec. 4, 2020
(86) PCT No.: PCT/IB2020/061515

§ 371 Date: Jun. 3, 2022

(30) Foreign Application Priority Data
Dec. 4, 2019 (ZA) 2019/08047

(51) Int. Cl. A61F 2/66; A61F 2/74

(52) U.S. Cl. CPC A61F 2/66; A61F 2/748; A61F 2002/6614; A61F 2002/5039

(57) ABSTRACT

The invention relates to prosthetic foot assembly comprising a prosthetic foot and a series elastic pneumatic actuator connected to the prosthetic foot, the series elastic pneumatic actuator being configurable between a first configuration in which the prosthetic foot is displaced to a first, rest position and a second configuration in which the prosthetic foot is displaced to a second position.

### FIELD OF INVENTION

This invention is in the field of prosthetic feet, in particular pneumatic actuated prosthetic feet.

### BACKGROUND OF INVENTION

The development of actuators is rapidly evolving and continuously new progress in terms of efficiency, power and force output is being reported. In other applications, pneumatics and hydraulics are rare in comparison with electrostatic, thermal and piezo-electric actuators. Studies have shown that pneumatic and hydraulic actuator delivers among the highest force and power densities at micro scale.

It is one of the objects of the present invention to provide an alternative actuator that can help during push off, by producing a force that will be sufficient to elevate and launch the centre of mass of the human user forward without the use of electricity.

It is of paramount importance to firstly understand the biological structures of the leg, kinematics and kinetics of these structures and how they interact during locomotion. Biological plantar flexors generate nearly 80% of mechanical work required to complete each gait cycle when walking on level ground and normally they produce more positive work than negative work. A normal body powered dynamic prosthetic feet, which are usually spring loaded using carbon fibre springs can only store and release mechanical energy while it's in contact with the ground and generate one half of mechanical energy. These prosthetic designs cannot generate a net positive work and can only release less than one eighth of mechanical power needed for propulsion from the biological plantar flexors.

The concept used in dynamic prosthetic feet that stores energy during foot flat and release this energy during push off is derived from the function of the Achilles tendon. It was categorised in the past that the tendon functions into two: tensile force transmission; and storage and release of elastic energy strain during gait. The elasticity of the Achilles tendon enables it to store and release mechanical energy, therefore decreasing the energy requirements for elevating and launching the centre of gravity during push-off.

The present invention intends to incorporate both the concept of energy storage and release with power production, which is what the series elastic element is all about. The series elastic elements is defined as every connective tissue in series with the contractile component, including the tendon. In below knee amputation, a portion of the series elastic element together with the ankle joint is removed. This disturbs the function of the series elastic element, as it can no longer bring about ankle joint displacement. Some of the prosthetic designs over the years have come up with a replacement of the series elastic element, by using a series of elastic actuators to bring about mechanical joint displacement. These series elastic actuators seem to work very well so far, they produce more positive mechanical work than negative work.

It is the object of the present invention to provide a type of a series elastic actuator that will produce more mechanical work, when used on a prosthetic foot, than those known in the art.

### SUMMARY OF INVENTION

According to the invention, there is provided a prosthetic foot assembly comprising:
a prosthetic foot; and
a series elastic pneumatic actuator connected to the prosthetic foot, the series elastic pneumatic actuator being configurable between a first configuration and second configuration in which the prosthetic foot is displaced between a first, rest position and a second position, respectively.

It will be appreciated that the first, rest position of the prosthetic foot may correspond with the configuration in which a plantar surface of the prosthetic foot is arranged substantially horizontal/parallel with respect to the ground, in use, and that the second position of the prosthetic foot may correspond with either the planar-flexion position of the prosthetic foot or the dorsi-flexion position of the prosthetic foot during a gait cycle, in use.

The angle defined by the prosthetic foot when configured between the dorsi-flexion and plantar-flexion positions during a gait cycle, in use, may be between 0 and 36 degrees.

The series elastic pneumatic actuator may comprise a pneumatic cylinder, preferably a single acting pneumatic cylinder, and an urging element, such as a spring, in-line or in-series with a piston extending from the pneumatic cylinder.

The prosthetic foot assembly may further comprise an air circuit comprising an air compressor in fluid flow communication with the series elastic pneumatic actuator for providing compressed air to the series elastic pneumatic actuator so as to enable the series elastic pneumatic actuator to be configured between the first and second configurations.

The air circuit may further comprise an air dryer, pressure gauge, a compressed air storage tank, a silencer, and a safety valve.

The air circuit may be connected to the series elastic pneumatic actuator by suitable conduits.

The prosthetic foot assembly may further comprise an exhaust valve to allow air to be released either to the atmosphere or the air storage tank or both, when the series pneumatic cylinder is configured between the first and second configurations. The exhaust valve may be fitted to the pneumatic actuator.

In an embodiment, in addition or optional to the air released back to the air storage tank being pressurized by the air compressor, the exhaust air may be arranged to be pressurized and compressed by the forces acting on the prosthetic foot during heel strike of the prosthetic foot in a gait cycle, in use.

The prosthetic foot assembly may further comprise a control valve, preferably a roller lever valve, which may be fitted to the prosthetic foot, the control valve being arranged to be opened when pressure/force is applied/exerted to the prosthetic foot, preferably exerted on the ball area of the prosthetic foot during a gait cycle, so as to allow compressed fluid (i.e. air) to flow from the air circuit into the series elastic pneumatic actuator to configure the series elastic pneumatic actuator between the first and second configurations.

The prosthetic foot assembly may comprise a support arrangement comprising: an operatively upper, upright member to which the pneumatic cylinder is connected;
an operative lower, moveable member that is moveably connected to the operatively upper, upright member;
a moveable connector, preferably a pivot connector, connecting the moveable member to the operatively upper, upright member;
a first base fixedly connected to the moveable member, the first base being accommodated in the prosthetic foot and being fitted to or proximate an
inner plantar surface of the prosthetic foot, wherein the first base has a distal end that is disposed proximate an operative front end of the prosthetic foot and a proximal end that is disposed proximate an operative rear end of the prosthetic foot; and
a second base that is spaced upwardly from the first base and disposed below the moveable connector, the second base extending away from the moveable member in the direction of the operative rear end of the prosthetic foot, wherein the urging element of the series elastic pneumatic actuator extends between the second base and free end of the piston that extends operatively in the direction of the plantar surface of the foot, such that, when the piston is displaced between a first configuration (i.e. rest position) and the second configuration (either extended or retracted position), the second base displaces the moveable member between a rest position in which the first base is in the first configuration which corresponds with the rest position of the prosthetic foot, and a second position in which the first base is displaced to a second configuration which corresponds with the second position of the prosthetic foot during a gait cycle, in use.

### BRIEF DESCRIPTION OF DRAWINGS

The objects of this invention and the manner of obtaining them, will become more apparent, and the invention itself will be better understood, by reference to the following description of embodiments of the invention taken in conjunction with the accompanying diagrammatic drawings, wherein:

FIG. 1 shows a cutaway view of a prosthetic foot assembly in accordance with the invention;

FIG. 2 shows the prosthetic foot assembly of FIG. 1 in a plantar-flexion configuration; and

FIG. 3 shows the prosthetic foot assembly of FIGS. 1 and 2 in a dorsi-flexion configuration.

### DETAILED DESCRIPTION OF AN EXAMPLE EMBODIMENT

The following description of the invention is provided as an enabling teaching of the invention. Those skilled in the relevant art will recognise that many changes can be made to the embodiment described, while still attaining the beneficial results of the present invention. It will also be apparent that some of the desired benefits of the present invention can be attained by selecting some of the features of the present invention without utilising other features. Accordingly, those skilled in the art will recognise that modifications and adaptations to the present invention are possible and can even be desirable in certain circumstances, and are a part of the present invention. Thus, the following description is provided as illustrative of the principles of the present invention and not a limitation thereof.

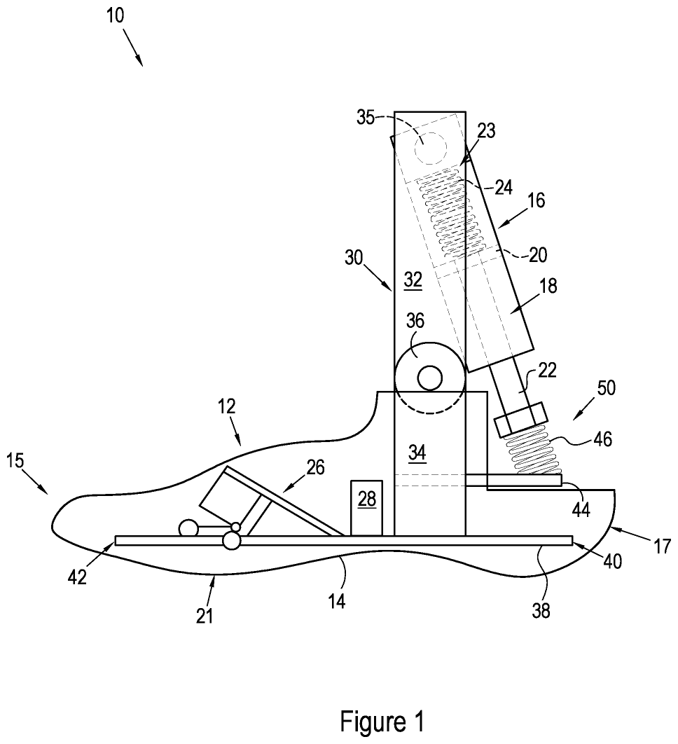

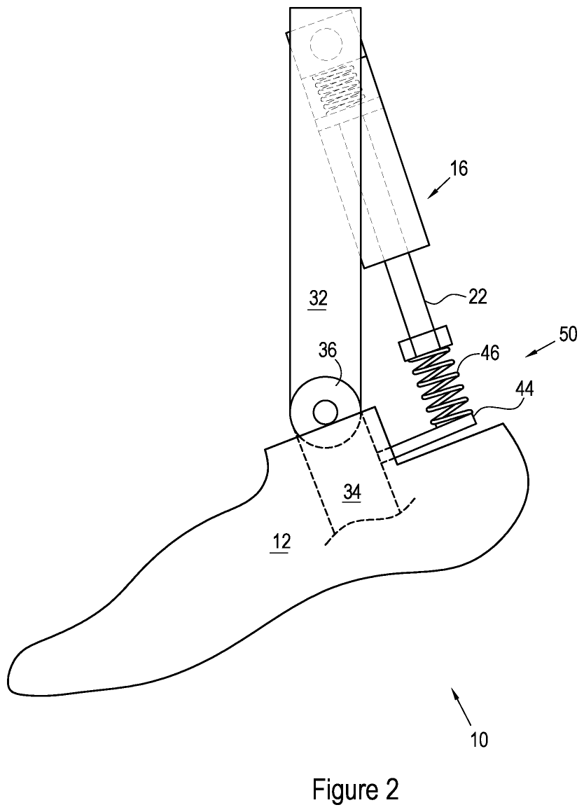

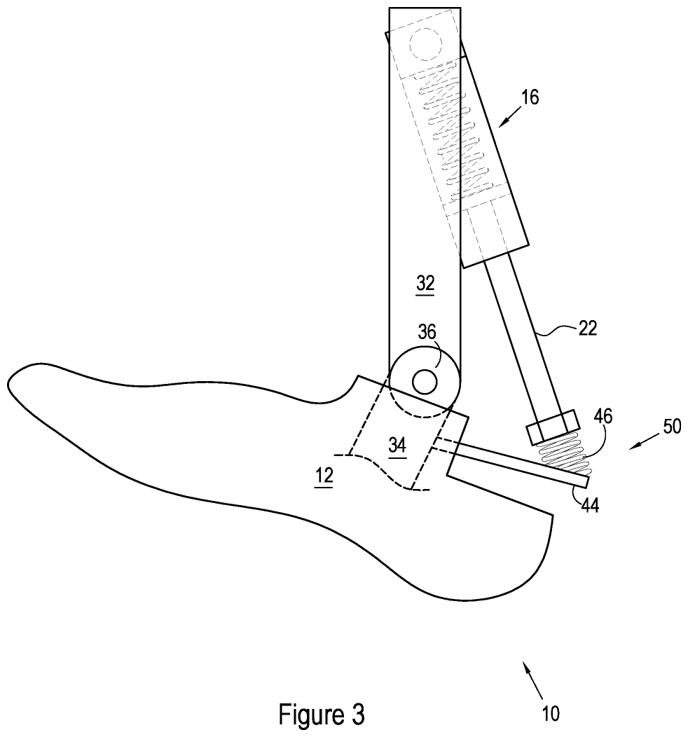

As can be seen in the drawings, there is provided a prosthetic foot assembly designated generally by reference numeral 10. The prosthetic foot assembly 10 comprises a hollow prosthetic foot 12. The prosthetic foot 12 is arranged to be displaced between a first, rest position, as shown in FIG. 1, in which a plantar surface (i.e. sole) 14 of the prosthetic foot 12 is arranged substantially flat/parallel with respect to the horizontal and can be configured to a second position which corresponds with either a plantar-flexion position as shown in FIG. 2, or a dorsi-flexion position as shown in FIG. 3 in which the angle defined between the plantar-flexion position and dorsi-flexion position, during a gait cycle, is within a range of about 0-36 degrees.

Turning our attention to FIG. 1, the prosthetic foot assembly 10 further comprises a pneumatic actuator in the form of single acting pneumatic cylinder 16. The pneumatic cylinder 16 defines an air chamber 18 in which a piston head/plunger 20 is arranged to be slidably displaced, and a piston rod 22 connected to the piston head 20 extends outwardly from the pneumatic cylinder 16. A first urging element, in the form of a spring 24, is fitted between the piston head 20 and closed end 23 of the pneumatic cylinder 16, and is arranged to bias/urge the piston head 20 such that the rod 22 is urged or biased to the extended configuration. As can be seen in FIG. 1, the piston rod 22 operatively faces and extends in the direction of the prosthetic foot 12.

The prosthetic foot assembly 10 further comprises an exhaust valve (not shown), typically a poppet valve (not shown) fitted to the pneumatic actuator 16. The exhaust valve (not shown) is arranged to release air from the pneumatic cylinder 16 as the piston head 20 and piston rod 22 are displaced to an extended configuration, as will be described in more detail further below.

The prosthetic foot assembly 10 further comprises an air circuit (partially shown in the drawings) which comprises a compressor 28, an air storage tank (not shown), a pressure regulator (not shown), a safety valve (not shown), a silencer (not shown), and conduits (not shown) that interconnect the units of the air circuit (not shown) and are arranged to transport compressed air to the pneumatic cylinder 16 and further transport exhaust air (i.e. the air released from the exhaust valve (not shown)) back to the compressor 28 and/or air storage tank (not shown) and/or atmosphere during a gait cycle, in use. The air circuit (not shown) is accommodated in the hollow prosthetic foot 12.

The prosthetic foot assembly 10 further comprises a control valve 26, in the form of a roller lever valve, which is fitted in the prosthetic foot 12. The control valve 26 is arranged to, when pressurized, as will be described below, allow compressed air in the air circuit (not shown) to be discharged into the pneumatic cylinder 16 so as to displace the piston head 22 to the retracted configuration to enable the prosthetic foot 12 to be displaced from the first, rest position, as shown in FIG. 1, to the plantar-flexion position, as shown in FIG. 2.

The prosthetic foot assembly 10 further comprises a support arrangement 30 comprising an operatively upper, upright member 32 to which the pneumatic cylinder 18 is fixed by a fixing element 35. The support arrangement 30 further comprises an operatively lower, moveable member 34 that is moveably connected to the operatively upper, upright member 32 by means of a pivot joint 36.

The support arrangement 30 further comprises a first base 38 that is fixedly connected to an operatively lower end of the moveable member 34, the first base 38 is accommodated in the prosthetic foot 12 and is connected to an inner surface of the plantar surface 14 of the prosthetic foot 12. The first base 38 comprises a proximal end 40 that is disposed proximate a rear end or heel 17 of the prosthetic foot 12, and comprises a distal end 42 that is disposed proximate a front end 15 of the prosthetic foot 12.

The support arrangement 30 further comprises a second base/pedal 44 that is spaced upwardly from the first base 38 and disposed below the moveable connector 36 and extends away from the second member 34 in the direction of the rear end 17 of the prosthetic foot 12. A second urging element 46 is connected in series with the piston rod 22 and accordingly extends between a free end of the piston rod 22 and the second base 44. In the context of specification and as is known in the art, the combination of the pneumatic actuator 16 and the second urging element 46 that extends from the piston rod 22 of the pneumatic actuator 16 is referred to as a series elastic pneumatic actuator and is designated herein by reference numeral 50.

In use, during a gait cycle, the prosthetic foot 12 is first arranged in the first, rest position as shown in FIG. 1 in which the series elastic pneumatic actuator is at a rest configuration. When pressure is exerted on the ball area 21 of the prosthetic foot 12 during the initial phases of lift-off or push-off of the prosthetic foot 12 from the ground or underlying surface (not shown), the control valve 26 is opened/triggered to allow compressed air in the air circuit (not shown) to flow into the pneumatic actuator 16. Once the compressed air is received in the chamber 18, the compressed air urges the piston head 20 in the direction of the closed end 23 of the pneumatic actuator 16 and simultaneously displaces the piston rod 22 inside the chamber 18 so as to configure the pneumatic actuator 16 in a retracted position, whilst at the same time the second urging element 46 is pulled in the direction of the pneumatic actuator 16. The tension applied to the second urging element 46 as it is pulled displaces the second base 44 in the direction of the pneumatic actuator 16, causes the moveable member 34 to pivot about the pivot joint 36 in the direction of the rear end 17 of the prosthetic foot 12. The displacement of the moveable member 34 further causes the first base 38, in particular the proximal end 40 of the first base 38 to also be displaced in the direction of the pneumatic actuator 16, so as to arrange the prosthetic foot 12 in a second, plantar-flexion position.

When the front end 15 of the prosthetic foot 12 lifts-off the ground (not shown), the pressure on the ball area 21 of the prosthetic foot 12 will diminish and this will trigger the closing of the control valve 26. At the same time, whilst the prosthetic foot 12 is in mid-air, exhaust air will be released from the pneumatic actuator 16 through the exhaust valve (not shown) as the piston rod 22 is displaced by the first urging element 24 to a rest configuration, as shown in FIG. 1, and the prosthetic foot 12 will be accordingly returned and reconfigured to the first, rest position, as shown in FIG. 1.

During heel strike in the same gait cycle, i.e. as the rear end/heel 17 of the prosthetic foot 12 engages the ground or underlying surface (not shown), the second urging element 46 will be placed under compression on the second base 44, and the piston rod 22 will be urged slightly into the chamber 18. Simultaneously, the second base 44 will be urged downwardly so as to displace the moveable member 34 in the direction of the front end 15 of the prosthetic foot 12 and accordingly displace the first base member 38, in particular the distal end 42 thereof, upwardly in order to arrange the prosthetic foot 12 in the dorsi-flexion position, as shown in FIG. 3.

It will be appreciated that the energy absorbed by the second urging element 46 during heel strike will be used to minimise the energy required during push-off of the prosthetic foot 12 in the subsequent gait cycle.

Also, during the heel strike, the air exhausted from the pneumatic actuator 16 will be compressed by the forces acting on the heel 17 as the heel engages the ground/underlying surface, and the resultant compressed air may either be transferred to the compressor 28 for further compression before being stored in the storage tank (not shown) or the resultant compressed air may be fed directly into the storage tank (not shown).

The pressure of the air returning to the air storage tank (not shown) is typically regulated by the gas pressure regulator (not shown) that ensures that the pressure inside the air storage tank (not shown) is less than the pressure of the compressed air returning to the air storage tank (not shown) during the gait cycle.

The prosthetic foot assembly 10 can achieve a range of motion that is wider than the anatomical joint and other active prostheses. It was found that the prosthetic foot assembly 10 that comprised a pneumatic actuator 16 having a bore size of 30 mm and piston rod size of 25 mm could produce a torque of between about 5.84 to 8.92 N.m, when the pressure of the compressed air was about 30 psi, and could also produce a torque of between about 11.67 to 17.84 N.m when the pressure of the compressed air was about 60 psi. The torque produced by the prosthetic foot assembly 10 of the present invention is larger than that of an anatomical joint and produces a torque that far exceeds that of both anatomical joint and active prostheses that use carbon fibre springs and electric motors. The prosthetic foot assembly of the present invention outperforms all passive prostheses, because of its ability to produce a net positive torque during ambulation.

As mentioned previously, the angle formed between the dorsi-flexion and plantar-flexion positions of the prosthetic foot in a single gait cycle is in the range of between about 0°-36°. By using suitable software, it was found that the 0°-36° active range of motion of the prosthetic foot 12 of the present invention could be completed in 14 milliseconds, which is equivalent to a joint velocity 4.5 radians per second. This joint velocity exceeds the maximum joint velocity of 2.97 radians per second of the anatomical joint required for normal walking/ambulation. Also, as is known in the art, the active range of motion required for a biological foot in normal walking is 30°; however, the prosthetic foot assembly 10 of the present invention can reach 36° active range of motion of the prosthetic foot 12 which outperforms the active range of motion that can be achieved by biological foot.

1. A prosthetic foot assembly comprising:
a prosthetic foot; and
a series elastic pneumatic actuator connected to the prosthetic foot, the series elastic pneumatic actuator being configurable between a first configuration in which the prosthetic foot is displaced to a first, rest position and a second configuration in which the prosthetic foot is displaced to a second position.

2. The prosthetic foot assembly according to claim 1, wherein the series elastic pneumatic actuator comprises a pneumatic cylinder, preferably a single acting pneumatic cylinder, and an urging element, connected to a piston extending from the pneumatic cylinder.

3. The prosthetic foot assembly according to claim 2, wherein the prosthetic foot assembly comprises an air circuit comprising an air compressor in fluid flow communication with the series elastic pneumatic actuator for providing compressed air to the series elastic pneumatic actuator so as to enable the series elastic pneumatic actuator to be configured between the first and second configurations.

4. The prosthetic foot assembly according to claim 3, wherein the air circuit is connected to the series elastic pneumatic actuator by suitable conduits.

5. The prosthetic foot assembly according to claim 4, further comprising an exhaust valve to allow air to be released either to the atmosphere or an air storage tank of the air circuit or both, when the series elastic pneumatic actuator cylinder is configured between the first and second configurations.

6. The prosthetic foot assembly according to claim 3, further comprising a control valve, preferably a roller lever valve, which is arranged to be opened when pressure is exerted to the prosthetic foot during a gait cycle, so as to allow compressed air to flow from the air circuit into the series elastic pneumatic actuator to configure the series elastic pneumatic actuator between the first and second configurations.

7. The prosthetic foot assembly according to claim 2, further comprising a support arrangement comprising:
an operatively upper, upright member to which the pneumatic cylinder actuator is connected;
an operatively lower moveable member that is moveably connected to the upright member;
a first base connected to the moveable member, the first base being accommodated in the prosthetic foot, wherein the first base is displaceable by the series elastic pneumatic actuator between a rest position which corresponds with the rest position of the prosthetic foot, and a second position which corresponds to the second position of the prosthetic foot.

8. The prosthetic foot assembly according to claim 7, wherein the support arrangement further comprises a second base that is operatively spaced upwardly away from the first base and disposed below a moveable connector that connects the moveable member to the operatively upper, upright member, and wherein the urging element extends between the second base and the piston that extends operatively in the direction of the plantar surface of the foot.

9. The prosthetic foot assembly according to claim 1, wherein the first, rest position of the prosthetic foot corresponds with the configuration in which a plantar surface of the prosthetic foot is arranged substantially parallel with respect to the ground, and the second position of the prosthetic foot corresponds with either the planar-flexion configuration of the prosthetic foot or the dorsi-flexion configuration of the prosthetic foot during a gait cycle, in use.

10. The prosthetic foot assembly according to claim 9, wherein the angle defined by the prosthetic foot when configured between the dorsi-flexion and plantar-flexion positions during a gait cycle, in use, is within a range of about 0° to 36°.
